(19) United States
(12) Patent Application Publication
(10) Pub. No.: US 2008/0020163 A1
(43) Pub. Date: Jan. 24, 2008

(54) Self-Adhesive Security Seal Sticker with an Incorporated Graphite or Granulated Layer for Fingerprint and Dna Collection, Procedure of Lamination to Manufacture This Sticker

(76) Inventor: Eduardo Luis Salva Calcagno, Buenos Aires (AR)

Correspondence Address: JACOBSON HOLMAN PLLC, 400 SEVENTH STREET N.W., SUITE 600, WASHINGTON, DC, 20004, US

(21) Appl. No.: 11/596,976

(22) PCT Filed: Apr. 27, 2005
(86) PCT No.: PCT/US05/14549

§ 371 Date: Feb. 20, 2007

(30) Foreign Application Priority Data
May 19, 2004 (AR) PO4010173

(51) Int. Cl. A61B 5/117

(52) U.S. Cl. 428/40.1; 427/11

(57) ABSTRACT

A self-adhesive sticker that comprises a kit of four superposed layers. The first layer provides the base, support, the second layer (3) is located in the middle of the kit, the third layer is made of graphite or granulated platte (10) placed on the upper surface of layer (3), and the fourth layer is a protective layer located on the upper surface of the kit.

### BACKGROUND OF THE INVENTION

1. Field of the Invention

This invention is related to a security seal to print, record or adhere fingerprints and DNA from people. An object of the present invention is to provide a method for prevention and deterrence of identity fraud, a security system to confirm identities, and a procedure to verify signatures or credentials that have been presented. A database containing the recorded fingerprint and DNA can be built. This is possible by printing the fingerprint on the seal's adhesive. Printed on the seal's adhesive also are the remains of epithelial cells and other organic debris, such as the finger's moisture, oil content, etc., which adhere to the skin surface and can be used in DNA testing by using certain reactive agents in an adequately equipped laboratory. The proposed sticker has four superposed components as a kit, including: an OPP (polypropylene) layer or base paper holding the whole kit at the bottom; an adhesive plate in the middle with adhesive on both of its upper and lower surfaces, incorporating on its upper surface a graphite or laminated plate, which can occupy half or more of the surface; and, finally, a highly resistant protecting sheet on the top of the kit. The seal provides a double security feature—the person's fingerprint and DNA. The seal is applicable to procedures to obtain a product for the asset legitimization, identity preservation, authentication of signatures, credentials, identity documents and any type of forms, with the object of avoiding fraud, use of adulterated documents or identity tampering in any operation and/or transaction.

2. Description of the Prior Art

Establishing an identity is the determination of a series of characteristics that distinguish an individual from the rest. Since ancient times, human beings have endeavored to establish an identification system that could tell people apart. Through research, a series of individual characteristics and singular data, both visible and internal, have been found to be useful in identity recognition. The fingerprints and DNA are identity characteristics we carry on our fingers and in our cells.

Identification through fingerprinting has been around for centuries. The use of this technique as a univocal means of identification goes back to the second century BC in China, where the identity of an important document's sender was verified by fingerprinting on wax. In the seventeenth century, it was known that fingerprints could be used to accurately identify an individual.

The nineteenth century saw the introduction of Henry's systematic classification system, based on patterns such as loops and circles, until the Austro-Hungarian national Argentinean Juan Vucetich was able to introduce a system of fundamental groups and sub-classifications, which is the system currently used to organize fingerprint files.

Today, we still use the traditional process of inking the finger and capturing the fingerprint on paper.

Moreover, the last years have shown that the digital scanning of these fingerprints has been the most successful biormetric system. The digital recognition of fingerprints makes up 80 percent of biometric systems.

There are many ways to execute an identification process. The most common method involves the capture and comparison of the points on a fingerprint called ‘minutiae’. Minutiae are those points where lines join or end.

Minutiae are considered the univocal characteristic of fingerprints.

Despite the different identification methods based on fingerprints currently available, all of them belong to a science known as Dactyloscopy, whose purpose is to propose the identification of individuals through the impressions left by the papillary crests of the fingertips.

All dactyloscopic systems are based on three fundamental principles, which are, Perenniality, Immutability and Diversity.

Perenniality: The fingerprints are perennial because the crests of the dactylar design are formed on the 14^{th} week of life of the fetus, and participate in the individual's growth until death.

Immutability: The fingerprints are immutable because dactylar images do not vary in terms of their individual characteristics, and because they are not affected by pathological phenomena. In case of the voluntary abrasion of the epidermal tissue, it regenerates in approximately fifteen days, showing again the original design.

Diversity: The fingerprints are diverse in shape because of the numerous and whimsical drawings the papillary crests can make, and because of the characteristic points distributed in dactylograms, making them unique. To date, no two identical fingerprints have been found.

The skin on the human body does not present a smooth surface. It has many rough areas that form dermal papillae, which sweat constantly. Therefore, it is considered that any area of the body that touches an ideal surface, especially if these are the fingertips or the palms of the hands, will leave the print of its dactylar or palm or plantar papillae, respectively, which are made of projections and depressions.

The projections are called papillary crests, and the depressions, inter-papillary. grooves. In the borders or vortexes of the papillary crests we find the sweat pores, where a fluid coming from the sweat glands, commonly known as sweat, is secreted. This fluid forms fingerprints, invisible to the naked eye but visible through a reactive agent to observe the dactylar image.

So far, we can conclude that fingerprints are a set of dactylar papillae, which form whimsical drawings on the fingertips, and when pressed on certain objects, they print their images through sweat secretion or ink-based coloring substances.

Generally, latent prints, whether invisible and revealed through reagents or inked, show dark or colored lines that represent the dactylar crests and white lines that represent inter-papillary grooves.

However, and as we shall see later, fingerprints cannot only be detected through the manual contact or manipulation of a certain object or surface. It is also possible to lift dead cells that can be useful in DNA analysis, complementing and enriching the process of identification for an individual. In fact, the fingerprints we leave everywhere when we touch the objects that surround us contain enough DNA to make the collection of our genetic codes from it possible.

Like the traces left by our fingertips, genetic digital prints are being used with increasing frequency in police investigations to identify criminals. These prints are obtained from samples of blood, semen, skin, hair, etc., which have been collected at the crime scene. Thanks to the analysis of the DNA contained in those samples, it is possible to obtain a genetic code, which, like a fingerprint, distinguishes an individual from the rest.

In this field, science has advanced by leaps and bounds during the last years. Researchers have been able to carry out experiments with amazing results. One of these experiments was to pass an item like a ball-point pen, a cup and a knife with plastic handle through the hands of several people. Scientists found out that the DNA contained in traditional fingerprints not only belonged to the last person who touched the objects. In addition, there was genetic material from then other people who had manipulated them. Moreover, the greatest amount of DNA found did not always correspond to the last person who touched the objects. Researchers also discovered that, in some cases, one nanogram (a millionth of a gram) of DNA was enough to be able to identify an individual.

Another experience demonstrated that, if there were two people holding hands for one minute, the DNA on the skin surface of one of them could be transferred to the skin of the other person. As expected, clean, dry hands leave the least DNA.

Current Issues With The Use of Ink to Capture Fingerprints:

Before fingers are inked, we must make sure that they are clean, dry and, above all, free from oils or sweat, to get good ink adherence on the ridges.

A dactylar print or fingerprint is defined as a replica of the fingertip's design on a fine cardboard, which is done by inking the fingertips on a finely polished iron plate previously smeared with an oil-based ink, by means of a rubber roller until there is a very thin, even layer. The next procedure is as follows. You take the right thumb of the person that will be fingerprinted between your left thumb and middle finger and press on the nail with your index finger. Then the finger that will be inked is placed on the plate, making contact first with the internal side and then rotating, it from right to left. Once the rotation is finished, the finger is withdrawn without making it slip on the plate.

To ink the rest of the fingers of the right hand (index, middle, ring and pinky fingers), you take each of them with your left thumb and middle finger around the existing auricular fold between the proximal and intermediate phalanges and press with your index finger on the fingernail. The finger to be inked makes left lateral contact with the ink plate and both finger and plate turn together from left to right, in order to ink the fingerprint from one end to the other. Once the rotation finishes, the finger must be withdrawn without slipping on the plate.

Once inked the fingers of the right hand will reproduce the fingerprints on the boxes labeled 1, 2, 3, 4 and 5 on the fingerprint card, in the same order in which they were inked, trying always to reproduce the flexion fold. To this effect, the fingerprint card is placed on a small 20 cm×12 cm×0.5 cm support board.

The card, shaped like a “U”, is folded. Therefore, boxes 1, 2, 3, 4 and 5, corresponding to the right thumb, index, middle, ring and pinky fingers, are on the foreground. Finally, we reproduce the fingerprints, beginning with the thumb and ending with the pinky. Then fingers 2, 3, 4 and 5 are joined to reproduce a joint image on the lower right corner of the card, providing proof that this is the natural order of the fingers. Sudden or brusque shifts must be avoided when withdrawing the support card that supports the fingerprint card. Then this burdensome operation is repeated for the left hand.

Please note that the thumb print in the lower portion must be done in an area indicated to that effect, with no rotation. Such an image provides the opportunity to appreciate, in greater detail, the areas commonly left at the crime scene, and also a better observation for classification, avoiding a proliferation of samples on the fingerprint form.

Besides the evident discomfort of the individual being fingerprinted and the lack of hygiene during the operation described above, there are other issues that may arise due to a deficient inking of the fingertips. This is a situation that often happens, given the complexity of the operation. These are some examples:

1. Pasted: The crests and troughs are pasted because there is too much ink on the roller or the crystal plate.

2. Illegible: At the time the inked fingertip prints the fingerprint there is a shift or slide, causing a smudge or a blurred, image. This can also happen if there is too little ink on the crystal plate.

3. Incomplete: During the inking the fingertips are not covered in their entirety, including all regions, both lengthwise and widthwise).

4. White spots: There is oil, dust, sweat, paint, etc. on the fingertips, so during inking they do not retain the ink pigment. For the operation to be effective, the individual must wash his or her hands with soap or gasoline before inking.

5. Image superimposition: When, during a faulty operation, the fingerprint is taken two or more times on the same box.

6. Different tonalities: When the inking is performed by pressing the roller directly on the fingertips.

7. Ink composition: Ink is a complex compound, more or less consistent and diversely colored when used for fingerprinting. In the current formula, inks are made of conductors, that is, by a group of substances that serve to transport coloring matter, as well as for solubility and drying characteristics. From a chemical point of view, these conductors are constituted by a greasy substance, a wetting agent, a solvent for the coloring compound, different additives and volatile liquids, black pigment of animal or plant origin, and other coloring agents, mostly acid. This ink past includes a solvent, colorants, and a thickening agent or plasticizer.

In addition to the aforementioned inconveniences, there is a series of conditioning factors during the inking operation that have to be taken into account to perform the fingerprinting:

1. A table with a marble, metal or glass plate;

2. A small deposit for printing ink;

3. Vertical compartments for the cards that have to be used during the operation;

4. A rubber or gel roller;

5. A small plate covered in zinc, with a wooden base and handles;

6. A grooved piece of wood to place the cards while the fingerprints are being taken;

7. Necessary elements to clean the individual's hands and/or fingers, and solvents to clean the tools used.

According to what has been detailed before, it is then clear that, today, fingerprinting is still problematic with the use of ink at present time. It is an archaic, difficult system that can be psychologically aggressive to the person being fingerprinted. In some cases, there is even the probability that the individual to be identified have allergic reactions to the chemical compounds being used to make the inks used in fingerprinting.

In addition, the inking procedure only permits the reproduction of a fingerprint image, since it cannot record the thickness of the epithelial lines. Thus, while measuring the differences between lines, important mistakes are made, since the image is flat and cannot reflect the actual depth or relief.

### SUMMARY OF THE INVENTION

To overcome these problems, a security seal of this invention proposes a new way of taking fingerprints, without the use of oil-based or paste inks, avoiding the use of chemical compounds and using instead a graphite plate incorporated into the security seal. There are several very important advantages regarding the use of this method to identify individuals. It is non-invasive. It obtains better-defined fingerprints than with inking. It allows obtaining the relief of the fingerprint (in addition to the image itself). It facilitates obtaining the DNA of the individual by lifting dead epithelial cells, which stick to the seal, and has incorporated security measures to avoid fingerprint fraud or forgery, preventing that one fingerprint being reused.

This invention is developed from to the previously known art by solving the issue of fingertip inking, adding at the same time an additional method that can be used to identify individuals: DNA obtained by dead cells that stick to the adhesive and/or granulated plate of the security seal. The present invention differentiates itself from all previously known inventions by the fact that it solves, in one configuration, issues related to the protection, safety and inviolability of the collected fingerprint and DNA, as will be shown later on when the invention is described in detail.

All these advantages are not covered, even minimally, by the known background of the nearest art.

To this effect, we can refer to U.S. Pat. No. 6,659,038 by the same inventor, patented Dec. 9, 2003.

This patent shows a security seal made of three superposed components: a paper base that will hold the whole kit, a central plate with adhesive on both sides and, finally, a highly resistant protective film.

However, said configuration only enabled citizens to leave a latent print, captured on the seal by an adhesive and which could only be lifted with a reagent. In addition, there was no additional security layer to avoid tampering with the seal, such as removing it from the form and using the same print on another form with another name.

Therefore, the purpose of this invention is to provide a self-adhesive security seal with incorporated graphite or granulated plate for fingerprinting and DNA collection. The present invention solves existing problems for the previous art with respect to the inking of fingers, print relief, collection of DNA samples, the differentiation between simple and organic prints and additional security measures to avoid tampering.

The process to make this self-adhesive sticker with security seal demands a special lamination process, because it cannot be manufactured through the conventional means of the technique. Therefore, another object of this invention is to present a lamination procedure that will be used in the manufacturing of a self-adhesive sticker with a security seal, with an incorporated graphite or granulated plate for fingerprinting and DNA collection.

### BRIEF DESCRIPTION OF THE DRAWING

FIG. 1 is an exploded view of the self-adhesive sticker with security seal for the present invention, which shows different layers that compose it.

FIG. 2 is a top plan view of the sticker, ready to be used.

FIG. 3 presents a perspective view showing how the sticker should be separated from the base layer.

FIG. 4 is a top plan view of the sticker, already on the surface where it will be used—a form, document, minutes, folio, etc.

FIG. 5 is a perspective view that shows how the sticker's protective film is to be separated, exposing the middle layer.

FIG. 6 presents a perspective view of the sticker with the protective film completely separate, exposing the middle layer that contains the graphite or granulated plate, according to the invention.

FIG. 7 illustrates the moment when a user rubs his/her finger on the graphite or granulated plate and leaves a fingerprint on the central plate.

FIG. 8 shows a perspective view of the security seal being closed to protect the fingerprint that has just been imprinted.

FIG. 9 is a top plan view detailing how the closed seal looks, with the protective film in sight.

FIG. 10 shows a perspective view of what would happen if someone wants to remove the security seal once it has been glued to a form or document, leaving in plain sight the security print transferred to that form or document.

FIG. 11 shows a perspective view of a second embodiment of the sticker, indicating where the protective film is separated to expose the graphite plate.

FIG. 12 details the next step of this second embodiment of the invention, showing how the graphite plate is separated to be rubbed into the corresponding finger.

FIG. 13 shows a perspective view of a user leaving his/her fingerprint on the sticker's central plate.

FIG. 14 is a top plan view of this second embodiment, with the protective film closed to protect the fresh fingerprint.

FIG. 15 is a top plan view of a third embodiment of the sticker with a structure similar to those of FIGS. 11 and 14, but with a transparent protective film, which allows seeing the fingerprint that remains.

FIG. 16 is a top plan view of a fourth embodiment, which serves to obtain fingerprints on site.

FIG. 17 shows a perspective view of how the protective film is separated from an opaque frame.

FIG. 18 shows a perspective view of the user leaving his/her fingerprint on the central plate of this fourth embodiment.

FIG. 19 is a top plan view of the fourth embodiment with the protective film closed, protecting the fingerprint.

FIG. 20 is a schematic view showing an overlapping of the layers that conform the final product, to manufacture this self-adhesive sticker with security seal.

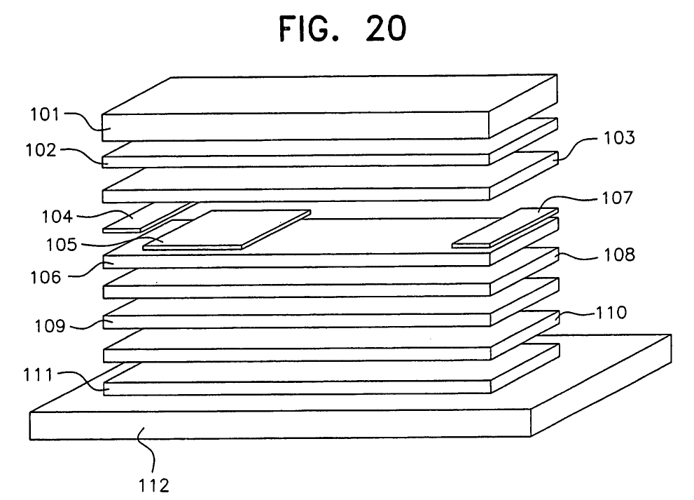

Components of a sticker with incorporated graphite or granulated plates in FIG. 20:
100: the surface for the text to be printed on the sticker.
101: Paper layer, preferably high-resistance, supercalendered, glassine kraft paper.
102: Polypropylene (OPP) layer (layers 101 and 102 are glued with the application of an aqueous acrylate PSA adhesive (not shown).
103: Silicone lamination.
104: Stripes on the 103 lamination, on the lower face, with no adhesive on the left border.
105: Graphite or granulated plate layer.
106: Lamination of Acrylate adhesive applied on the upper face of layer 108.
107: Stripes on the 106 lamination, on the upper face, with no adhesive on the right border.
108: Polypropylene (OPP) layer.
109: Lamination of Acrylate adhesive applied on the lower face of layer 108.
110: Adhesive lamination with security imprint.
111: Silicone lamination.
112: Polypropylene (OPP) layer.

### DESCRIPTION OF THE EMBODIMENTS

The procedure to use the sticker is described below. First, a finger that will be used to obtain a visible fingerprint is selected. To this effect, that finger is rubbed on a graphite or granulated plate of the self-adhesive sticker to get a transfer of graphite from the graphite plate to the finger in question, impregnating and covering most of the fingertip area. Once the finger is impregnated with dry graphite powder from the plate, this finger will be lightly pressed on the adhesive surface of a two-sided layer. The reaction between the adhesive, which is moist, and the graphite powder in the finger, which is dry graphite, is as follows. The adhesive retains the graphite powder that adhered to the surface of the external papillae (crests) of the finger impregnated with graphite. When the finger is pressed on the adhesive surface of the layer, it will leave the image of the desired fingerprint, which will be a clear representation of the original, with no deformations, to identify the person who imprinted it according to the dactyloscopic system used for classification. In this way, we avoid the intermediate papillary grooves between the crests getting clogged, which happen sometimes with the wet or paste ink traditionally used to get fingerprints.

A very important aspect of this procedure should be noted. When dry graphite powder is transferred from the impregnated finger to the adhesive where it is retained, the graphite is practically cleaned off the finger. This is another marked advantage of this invention, not previously evident for a person of ordinary skill in the art. With this invention, not only the issue of document forgery or tampering is solved, the problem with inked fingers is solved as well, in addition to the possibility of obtaining the individual's DNA together with the fingerprint. The mechanics used, as described for the graphite plate, are the same for an alternative embodiment with a granulated plate instead of a graphite plate.

This invention, as previously explained, relates: to a security seal to imprint, register or glue the fingerprints of individuals. One object of the invention is to provide a method of prevention and deterrence of identity fraud, a security system to validate identities and a safe procedure to legitimize signatures or credentials on forms, documents, minutes, folios, etc. In this way a database of the fingerprints, including their relief, is captured on the seal's adhesive, as well as dead epithelial cells and organic remains (finger moisture, oils, etc.), so a DNA test can be performed in a laboratory with adequate reagents.

The self-adhesive sticker with security seal for fingerprinting and DNA collection is a security product that comes in plates, prepared for individual use and gluing on forms of the document that will contain it. In other words, it will replace the traditional empty space we see today on immigration forms or identity documents, where the fingerprints of the individuals are registered.

A summary of the possible uses for the sticker presented in this invention could include the collection of:

1. Fingerprints: to incorporate them to any form, such as “DACTYLOGRAMS”, which are used by the Ministry of Justice for the identification of individuals.

2. Plantar prints: for newborn templates, forms or templates made to that effect, to collect plantar prints from underage persons or persons of legal age on templates called “PODOGRAMS”.

3. Palm images: to register and archive individuals' ID (identification).

4. Accumulation of epithelial cells for DNA identification; archiving the person's biological and biometric data.

5. Any other forms with adhesive surfaces where biometric or biological data is to be collected from any citizen or individual, national or foreign.

The final product is highly functional, given its practical use. It is hygienic, because any remaining graphite disappears by rubbing the impregnated surface with toilet paper or even another finger. It is harmless, since it does not cause any allergies or adverse reactions of any kind. It is economical and versatile, because it adapts to the different needs of the user, according to different special or universal needs adopted for its final configuration.

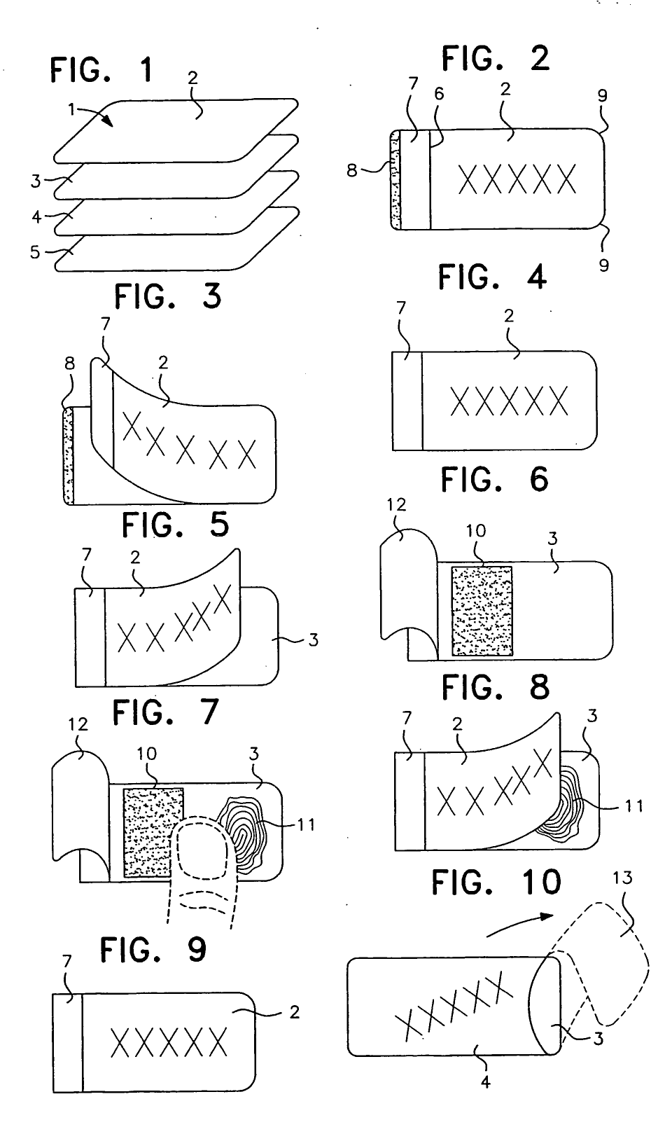

In the prefer embodiment (FIGS. 1 to 10), the self-adhesive sticker is made of mainly four plates, one on top of the other. We say “mainly four” because, as it will be explained in detail later on, some of these four plates are in turn made of several layers or sub-plates, though they are not visible to the user's naked eye. The first plate (5), starting from the bottom of the structure (FIG. 1), is called the base support. It is preferably a polypropylene sheet with a shiny, smooth finish and silicon treatment on the whole upper surface (5), and which is characterized by the following properties: thickness between 65 and 75 microns and highly resistant to longitudinal or transversal traction, since its function is to support the other plates that make up the security seal and to allow its separation.

This base plate (5) can be individual, as in the example shown, or it can be a sheet or roll that contains a certain number of self-adhesive stickers. In the example shown, the base plate (5) has the biggest surface in the whole kit, and it includes a rough tab (8) protruding a short distance, such as a couple of millimeters. The tab (8) can be used to support the base paper (5) while the rest of the kit is separated, as shown in FIG. 3.

The second plate (3) is located in the middle of the kit. It consists of a double-sided support material with adhesive on both sides. It will be preferably a polypropylene film of 10 to 20 microns thick with both sides treated with a PSA-type adhesive containing aqueous acrylate, which does not produce allergic reactions. It is not sealable by heat and is highly resistant to traction. This two-sided adhesive plate (3) has a weight of 11 g/m², a thickness of 12.5 microns, resists traction in the order of 1,400 kg/sq. inch and is preferably crystal white in color. On the upper surface of this two-sided adhesive plate (3), there is a non-adhesive border to the right, 5 to 7 mm wide, so the upper cover (2) of the seal can be lifted, as illustrated in FIG. 5. In certain embodiment, when plate 2 is transparent, we can do without the border according to the use given to the seal so the user's fingers do not stick to the adhesive.

The lower surface (13) of the two-sided adhesive plate (3) also contains adhesive and undergoes a security imprint treatment to prevent the transfer of self-adhesive sticker to another document or to be reused. FIG. 10 shows what happens if an incorrect user, tries to remove the self-adhesive sticker from a document: a graphic legend of security seal 4 will be indelibly associated with the document originally holding the sticker, demonstrating that there has been a breach or incorrect manipulation. In this way it is not possible to use the sticker a second time.

The security seal is a notable innovation with respect to the previously known art, because it incorporates necessary tools to prevent fraud, duplicity or identity forgery, etc. Today, with conventional documents or forms, it would be simple to replace one person's fingerprint and attach it to another's document. The third plate is composed of a graphite or granulated plate (10) approximately 10 mm wide, which is inserted on the left side of the upper surface of the two-sided adhesive plate (3). A user can rub a finger on the plate and then stamp the finger on the portion of the plate with adhesive, which is on the right side of the upper surface of the two-sided adhesive plate (3), leaving a visible or latent fingerprint with all organic components adhered to the plate. The adhesive used is preferably of PSA type, which is an acrylate copolymer in aqueous dispersion. It has good initial tack as well as good adhesion and cohesion characteristics, with a loop tack of 950 grams, an adhesion of 750 grams and cohesion of 24 hours once transplanted to a form or document.

The introduction of the graphite or granulated plate, according to the use given to the seal, into the self-adhesive sticker kit constitutes one of the most highlighted elements of this invention regarding its novelty and inventive activity, even if compared with a closer technique. Because of the particular characteristics of these plates, we will provide a detailed description of the graphite or granulated plates. These plates can be incorporated into the self-adhesive sticker kit individually, alone or together.

The fourth plate in the kit is a protective film (2), located on the upper side of the kit. It is preferably made of supercalendered, glassine kraft paper with high density and resistance, typically with a weight between 62 and 67 g/m², a thickness of 55.8 microns, a tear load of 17 kg and crystal white in color. This film can also be composed of polypropylene (OPP) material with a thickness of 50 microns. The upper surface (2) of this component has a crown treatment so the “security seal” denomination or a text string related to its use can be printed on it. The lower surface (12), which is in contact with the adhesive of the two-sided central plate (3), is covered by a very thin polypropylene film, with a border (7) on the left side to have a window or stop effect, as shown in FIGS. 7, 8 and 9.

The complete kit containing a self-adhesive sticker with security seal and incorporating a graphite or granulated plate (according to use) can have different shapes, sizes and designs. The preferred embodiment is the one shown, which is rectangular in shape, approximately 50-55 mm long and 25-35 mm wide, with no sharp corners. They have been nipped, so there is a 135° angle between any two adjacent sides.

In addition, according to specific user's needs, the proposed self-adhesive sticker can be manufactured under any possible lamination option. For example, the graphite plate can be on the right side instead of the left. The polypropylene (OPP) film can be transparent, or of different colors. Alternatively, the adhesive in the central plate can be colored, transparent, etc.

The second embodiment is illustrated in FIGS. 11 to 14. It comprises the same four plates as the preferred embodiment, the difference being that the graphite plate (10a) is not incorporated into the double-adhesive central plate (3a), but is a complete graphite plate that can be detached from the kit, used and discarded. Therefore, at first, the user will have to lift the protective film (2a), then detach the graphite plate (10a) using-a border (10b) to that effect, rub his/her finger on the detached graphite plate (10a) (not shown), and then imprint his/her fingerprint (11a) on the double-adhesive central plate (3a). Finally, the user can close the protective film (2a), putting its lower surface (12a) in contact with the central plate (3a) (fingerprint holder).

This lower surface (12a) that is in contact with the adhesive of the two-sided central plate (3a) is covered by a very thin polypropylene film with a border (7a) on the left side for stop effect, as shown in FIGS. 11 to 13. On the other hand, FIG. 14 shows the closed kit with an opaque protective film (2a) with instruction text covering and protecting the user's fingerprint.

This alternative embodiment of the invention also has a security printing treatment to prevent the self-adhesive sticker from being transferred to another document or reused, like in the preferred embodiment.

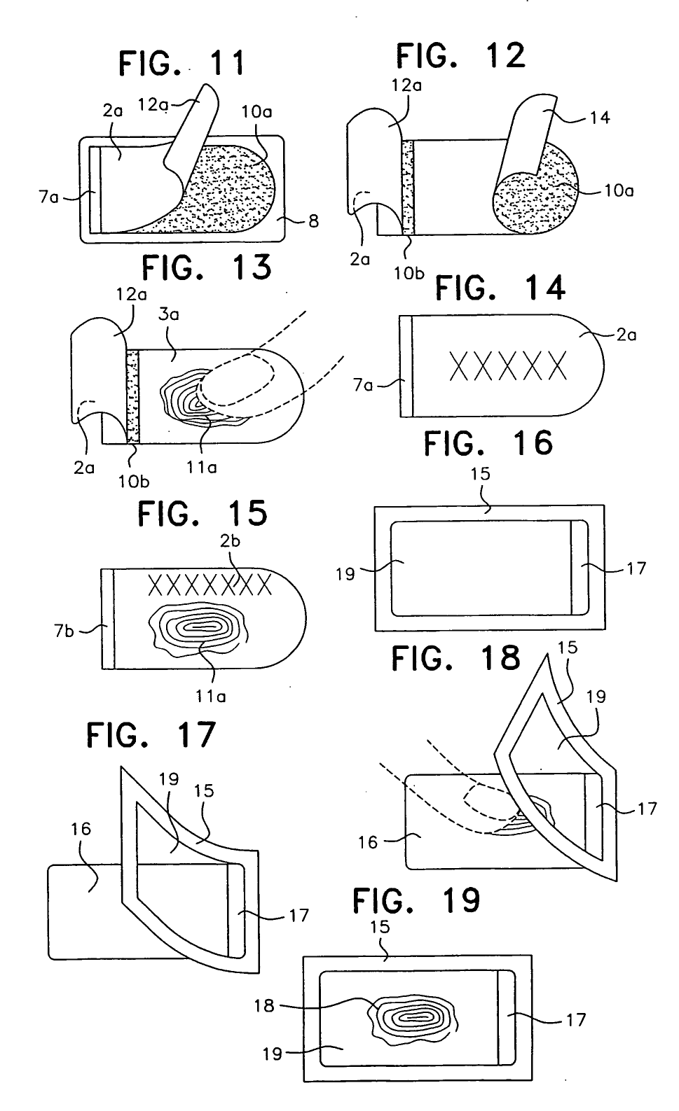

In addition, the invention includes a third embodiment for the self-adhesive sticker, as illustrated in FIG. 15.

This embodiment is generally the same as the previous one, the only difference being that in this case the protective film (2b) is transparent, so the user's fingerprint (11b) is visible.

This third alternative embodiment also has a security printing treatment to prevent the self-adhesive sticker from being transferred to another document or reused, like in the preferred embodiment.

In addition, the invention provides a fourth alternative embodiment of the self-adhesive sticker, illustrated in FIGS. 16 to 19.

This fourth embodiment has a different application compared to the previous ones. That is the reason why it has a different configuration. Whereas the first three embodiments are intended to be used on identification documents or forms, this alternative embodiment is designed to lift fingerprints from different surfaces, or to take fingerprints on site from individuals that need to be identified later on. For example, police personnel could use it to identify a person to verify his/her true identity, in case he/she is a suspect. This embodiment would also be very useful for security personnel, especially firefighters and police officers or coroners to obtain fingerprints and DNA from people who have suffered traffic accidents or have committed a crime and who are not carrying any ID documents.

This “portable” version of the sticker is not self-adhesive and only has two layers instead of the four in the other embodiment. In this case, the sticker includes a transparent protective film (19) with a surrounding opaque border (15) and a central plate with adhesive (16) with a border (17) on the right that allows it to be folded.

In this version, the middle plate (16) has on its lower surface (base) a polypropylene film with a smooth, shiny finish, while the upper surface (where the fingerprint will be imprinted) has an adhesive treatment similar to those previously described. The adhesive used is preferably of PSA type, which is an acrylate copolymer in aqueous dispersion.

The second plate of this portable embodiment of the sticker is the protective film (19), which has on its upper surface a polypropylene (OPP) film, transparent at the center with an opaque perimeter border (15). The lower surface, which will be in contact with the adhesive on the central plate, has a silicone treatment.

In case this embodiment of the sticker was used to lift latent prints from certain surfaces, this would be possible by lifting the transparent protective film (19) and pressing the adhesive surface (16) of the central plate onto the surface where the latent print is. On the other hand, if the sticker is used to determine the identity of a person on the street, it would be necessary to follow the procedure illustrated in FIGS. 16 to 19, with the exception that the print can be latent when pressing the finger of the individual that is to be identified on the central plate's adhesive, or it can be visible, as shown in FIGS. 18 and 19. In the latter case, the user will have to rub a loose graphite plate on the individual's finger, since the “portable” version does not incorporate it. To this effect, we provide a block of graphite plates of reduced dimensions (3 cm×4 cm, for example), which are glued on one end to form the block and which the user will have to carry for this on-site fingerprinting.

In an intermediate stage between the steps shown in FIGS. 17 and 18, a user detaches a graphite plate 10a from the sticker, rubs the finger of the individual to be identified, imprints his/her fingerprint on the middle plate's (16) adhesive then covers the fingerprint by the transparent film (19);

So far, we have explained in detail the configuration of the sticker, with all the plates that compose it in different embodiments. However, it is also necessary to know the specific composition and manufacturing process of the graphite plate or the alternative version, the granulated plate.

Detailed Structure of the Graphite Plate:

The graphite plate (10), which is incorporated into the two-sided plate (3) of the self-adhesive sticker or provided stand alone as in the last example, appears, at first sight, as a single layer. However, it is composed of three different sequentially superposed layers from the bottom to the top: the first layer is a layer of mechanical paper; the second layer is a silicone layer; and the third layer is a graphite layer.

Description of the Elements That Constitute the Graphite Plate:

The surface material, including the first and second layers, is a piece of mechanical paper with silicone covered on its upper surface, onto which powdered graphite (the third layer) is dusted. The mechanical paper is of glassine type with high density and resistance, with silicone on one surface giving it a smooth and semi-glossy finish. Typically, it has a weight between 75 and 85 g/m², which can vary according to the use, and a thickness between 65 and 75 microns. In addition, for the mechanical paper, the resistance to longitudinal traction is 15-20 kg/in, and the resistance to transversal traction is from 7.5 to 11 kg/in. The color of the mechanical paper is preferably white, and it comes in coils resting on bushings 70-75 mm in diameter.

The graphite of the third layer is a harmless organic component, and comes from graphite ground into a fine, talcum-like dry powder.

Manufacturing Process for the Graphite Plate:

In the lamination process, silicon is added to the mechanical paper layer, resulting in what is called a siliconed paper layer.

The ground, dry, talcum-like graphite powder is rubbed onto the siliconed paper, the graphite adhering to the siliconed paper due to static charge.

The siliconed paper layer with the adhered graphite becomes the graphite plate, which is then cut in the shapes needed according to the different uses.

We have provided the detail composition of the graphite plate, which, as said before, has as its function to replace the old technique of finger inking to take an individual's fingerprints. Now, it has already been mentioned that an object of this invention is not only to provide a self-adhesive sticker for fingerprinting, but also as a means to collect an individual's DNA via the organic debris (finger moisture, oils, etc.) and particles of dead epithelial cells left adhered to the plate. These organic components can allow DNA testing in an appropriate laboratory setting.

To this effect, it has been verified that providing a granulated plate with a coarse surface facilitates the collection of greater quantities of genetic information, because the dead cells and organic debris on the fingertips are easily lifted when they are subjected to slight friction, which would be the case with a coarse, granulated plate. That is why this invention proposes an alternative presentation of a self-adhesive sticker with the same structural features as the different variants we have described up to now. The only difference is the replacement of the graphite plate with a granulated plate, whose characteristics will be described below.

Detail Structure of the Granulated Plate:

The granulated plate (10), which is incorporated into the two-sided plate (3) of the self-adhesive sticker, appears, at first sight, as a single layer. However, it is composed of three different layers: the first layer is a layer of uncoated paper with a weight between 50 and 80 g/m²; the second layer is a layer of PSA adhesive; and the third layer is a layer of fine grains of pumice stone.

Description of the Components:

The surface material, including the first and second layers, is a piece of glued uncoated paper, onto which finely ground pumice stone, (the third layer) is dusted. The uncoated paper has high density and resistance, with adhesive on one face. Typically, it has a weight between 50 and 80 g/m², which can vary according to the use, and a thickness between 30 and 50 microns. In addition, for the uncoated paper, the resistance to longitudinal traction is 10-15 kg/in, and the resistance to transversal traction is from 7.5 to 10 kg/in. The color of the uncoated paper is preferably white. The paper is taken from coils resting on bushings of 70-75 mm in diameter.

The fine pumice stone grains of the third layer are inorganic component from pumice stone that has been ground into a fine, dry powder.

Manufacturing Process for the Granulated Plate:

In the lamination process, an adhesive is added to the uncoated paper layer, resulting in what is called a granulated pumice stone paper layer.

The finely ground, talcum-like pumice stone powder is sprayed onto the glued paper by means of a compressed air sprayer, producing a complete coating of granulated pumice stone powder sticking to the glued paper.

The granulated pumice stone paper layer is sent, as a granulated plate, to the cutting section, which produces the shapes needed by the different uses.

Graphite Layer:

When lifted, the components align themselves in laminas (self-adhesive laminating technique) with silicone strips. Together they form a two-sided self-adhesive, laminated coil with graphite strips. This coil is then inserted into a flexography machine with different types of rollers, until the coil is cut in the necessary shapes and sizes, according to the use characteristics of the product.

Granulated Layer:

When lifted, the components align themselves in laminas (self-adhesive laminating technique) with silicone strips. Together, they form a two-sided self-adhesive, laminated coil with granulated pumice stone strips. This coil is then inserted into a flexography machine with different types of rollers, until the coil is cut in the necessary shapes and sizes, according to the use characteristics of the product.

As it has been detailed in the process to manufacture graphite and granulated plates, to reproduce the invention, it is necessary to describe the procedure for the manufacture of the whole sticker, since it is an object of the present invention to provide a laminating procedure in order to manufacture this sticker.

Procedure to Manufacture the Self-adhesive Sticker With the Security Seal:

The laminating procedures needed to manufacture layers of the sticker can be carried out in different stages, without a preordained arrangement. However, the final assembly of the different laminations has to be performed at the laminating plant according to a predetermined sequence.

This sequence is established according to the different product uses, to satisfy different customer needs regarding the use and usefulness of the self-adhesive sticker with security seal.

That's why it is important to point out that the layer can have different processes and can be striped in different silicone or adhesive laminations on different surfaces and in different layers. This can also be inverted regarding the components of a layer.

In addition, according to the different use needs for the sticker, graphical imprints and visible security components can be added to the product in order to give it unique characteristics. The purpose of the above is to avoid that the layer or the final sticker be copied or plagiarized, since they are the final products obtained from each and every one of the lamination processes that have been carried out.

In this way we can obtain a layer, either with the incorporation of graphite or granulated plates or not, laminated with security imprints (to avoid the reutilization of the stickers) by means of adhesives or silicones, which, when exposed to ultraviolet light, highlight univocal security characteristics to make sure the final product is not plagiarized. Alternatively, we can incorporate the graphic security measures the customer wants to implement.

Processes For the Different Layers That Compose A Finished-product:

To create the final product shown in FIG. 20, it is necessary to develop a series of processes to make each layer that composes the multi-layered, final-product. These previous processes will be described below. Basically, there are four layers, one for each individual layer that composes the final-product.

1) Protective and External Layer

It comprises layers 100 through 104. It includes a super-calendered, glassine kraft paper layer (101) that-must undergo a previous crown treatment process (for ink fixation) on the external surface (100) that will be exposed. Different logos, numeration, etc. can then be printed on this external surface (100), with different inks and pigments that will not slide off.

The layer (101) undergoes, a gluing process on its internal face, to which a polypropylene layer (102) is glued. The polypropylene layer's lower surface receives a silicone treatment (103) in order to prevent the upper printed cover (101) from getting stuck on the two-sided plate (108).

The methods to apply either adhesive or silicone onto these layers are carried out with the conventional means.

The polypropylene layer (102) with silicone on its lower surface undergoes a striping process (104), in which a left border approximately 10 mm wide, does not receive any silicone. The purpose of this is to be able to have a window or stop effect, which is necessary to handle the sticker while opening it.

2) Graphite/Granulated Plate

The process to manufacture the graphite or granulated layer was already described above.

3) Two-Sided Central Plate

A polypropylene layer with a white crystal base undergoes a process in which adhesive is applied to both surfaces. The adhesive that is applied is preferably an acrylate-based adhesive. This layer also goes through a striping process, in which a 10 mm wide strip is left without adhesive on one side to be able to lift the protective layer from that side. In addition to the adhesive, the lower surface also goes through a graphical security imprinting process. The purpose is to avoid the reutilization of the seal. An imprint will be left on the original document or form, indicating that the seal was altered, if someone wanted to transfer and use it in another document. This is achieved through a treatment in which a hollow cylinder with the desired security text is pressed onto the adhesive. The adhesive will not be coating the text's surface at 100% effectiveness, but it will coat the non-text surface at 100% effectiveness. Therefore, if someone wants to lift the sticker from the form, the adhesive at non-text surface will remain adhered to the sticker, but the adhesive at the text surface will stay glued to the form to achieve the desired effect.

4) Base Layer

This polypropylene layer has silicone applied on one surface, where the self-adhesive sticker with the security seal is inserted, so the sticker can be easily lifted off the base layer and taken to be adhered onto a form or document.

Final Lamination for the Self-adhesive Sticker:

Once these four processes have been carried out on the different layers that have been previously described, the layers are taken to a final laminating process in which they are assembled in a pre-established sequence to obtain a final-product. This sticker's characteristics and different lamination layers are presented in a structural order, represented in FIG. 20, for the preferred embodiment of the self-adhesive sticker with security seal.

It is evident that one of the different layers that make up the lamination process can vary according to the different embodiments a user may want from the invention. For example, we have the alternative embodiment, described above. It provides a portable version of the sticker with a one-sided central plate with adhesive on only one of its surfaces. Therefore, the corresponding lamination process will vary with respect to the one shown in FIG. 20.

The layer obtained will be used as raw material to manufacture the sticker. This product is manufactures by means of a series of superposed lamination processes with a pre-established sequence according to the use and usefulness of the security seal. Therefore, their sequence or arrangement can change with the customer's needs.

During the process to put into practice the aforementioned and previously described self-adhesive sticker with security seal, as well as during the different stages that comprise the lamination procedure to manufacture said sticker, and the final-product that results from this procedure, modifications and variations may be introduced to the embodiment, all of which shall be deemed included within the scope of protection of the present invention.

1. A self-adhesive sticker with security seal and an incorporated graphite or granulated plate for fingerprinting and DNA collection, characterized in that the sticker comprises four superposed layers forming a rectangular kit, wherein:
a first layer is a base layer (5) located at the bottom of the kit for receiving other layers, and has a larger surface than other layers, and a border that protrudes beyond the perimeter of other layers, and a silicone treatment on its upper surface;
a second layer (3) is located on the top of the first layer and in the middle of the kit, and includes a support material with adhesive on both an upper surface and a lower surface (13), and an adhesive lamination with a security imprint on its lower surface (13) which is in contact with the upper surface of the first layer, wherein, after the lower surface of the second layer is separated from the upper surface of the first surface and adhered to a document surface, when the self-adhesive sticker is removed from the document surface, the security imprint will be left on the document surface;
a third layer includes a graphite or a granulated plate (10), placed onto the upper face of the second layer (3); and
a fourth layer includes a protective polypropylene film (2) located on top of the kit, which has a previous crown treatment on its upper surface, and has a lower surface in contact with the second layer (3) and the third layer, and a stripe (7) on one lateral border.

2. The self-adhesive sticker with security seal according to claim 1, characterized in that the first layer (5) includes a polypropylene film with a shiny, smooth finish on its lower surface.

3. The self-adhesive sticker with security seal according to claim 1, characterized in that the second layer (3) includes a polypropylene (OPP) film that has both an upper surface and a lower surface treated with an adhesive, preferably an aqueous acrylate of type PSA, and a non-adhesive stripe on one of the smaller lateral borders of the upper face.

4. The self-adhesive sticker with security seal according to claim 1, characterized in that the graphite plate (10) comprises three superposed layers: (a) a base layer made of mechanical paper; (b) a silicone application with a smooth, shiny finish; and (c) a layer with finely ground graphite; wherein the graphite plate (10) adheres to the second layer (3) by means of an adhesive, preferably an aqueous acrylate of PSA type.

5. The self-adhesive sticker with security seal according to claim 1, characterized in that the granulated plate (10) comprises three superposed layers: (a) a base layer made of uncoated paper; (b) an adhesive application, preferably of PSA type, and (c) a layer of finely ground pumice stone; wherein the granulated plate (10) adheres to the second layer (3) by means of an adhesive, preferably an aqueous acrylate of PSA type.

6. The self-adhesive sticker with security seal according to claim 1, characterized in that the protective polypropylene film (2) includes a layer preferably made of high-density, high-resistance supercalendered kraft paper.

7. The self-adhesive sticker with security seal according to claim 1, characterized in that the graphite plate (10a) includes a whole layer that can be lifted off the sticker kit using a stripe (10b).

8. A self-adhesive sticker with security seal for fingerprinting and DNA collection, characterized in that it comprises two superposed layers forming a rectangular kit, wherein a first layer on the top of the kit is a transparent protective film (19) with an opaque surrounding border (15); and a second layer at the bottom of the kit comprises a central adhesive layer (16) in the middle with a stripe (17) on one of the smaller lateral borders; and characterized in that:
the central adhesive layer (16) includes on its lower surface a film, preferably of polypropylene (OPP) with a smooth, shiny finish, and an upper surface of the central adhesive layer (16), where the fingerprint will be captured, has an adhesive treatment; and
the protective film (19) has a transparent polypropylene (OPP) film in the center of its upper surface, with an opaque surrounding border (15), and the lower surface of the protective film (19), which will be in contact with the adhesive of the central adhesive layer (16), has a silicone treatment.

9. The self-adhesive sticker with security seal for fingerprinting and DNA collection according to claim 8, characterized in that the adhesive treatment for the upper surface of the central adhesive layer (16) entails the application of an adhesive preferably of PSA type, an acrylate copolymer in aqueous dispersion.

10. The self-adhesive sticker with security seal for fingerprinting and DNA collection according to claim 9, characterized in that the sticker kit includes a block of graphite plates that are not incorporated into said sticker.

11. A method for manufacturing a self-adhesive sticker with superposed layers and security seal for fingerprinting and DNA collection, characterized in that the method comprises the following steps:
laminating a protective layer that comprises a sheet (101) of supercalendered kraft paper with an upper surface that undergoes a crown treatment for ink fixation and a lower surface that goes through an adhesive treatment, onto a first polypropylene sheet (102) having an upper surface and a lower surface that goes through a silicone treatment (103), wherein the lower surface of the sheet (101) of supercalendered kraft paper is glued to the upper surface of the first polypropylene sheet, and the lower surface of the first polypropylene sheet (102) additionally goes through a silicone application (103) and a stripe process (104) on one of its smaller lateral borders with no silicone application on that stripe;
laminating the protective layer onto a central layer that comprises a second polypropylene sheet (108), preferably with a crystal white base, which has an upper surface (106) and a lower surface (109) that both go through an adhesive application process, wherein the lower surface of the first polypropylene sheet (102) with the silicone application (103) of the protective layer is in contact with the upper surface of the second polypropylene sheet (108) of the central layer, the polypropylene sheet (108) of the central layer goes through a striping process (107) on its upper surface (106) where the adhesive is not applied on one of the smaller lateral borders and the lower surface (109) of the second polypropylene sheet (108) of the central layer goes through a security imprinting process (110), in which a hollow cylinder with the desired security text is pressed onto the adhesive so that the adhesive will not be coating the text's surface at 100% effectiveness, while the non-text surface is coated at 100% effectiveness; and
laminating the central layer onto a base layer that comprises a third polypropylene sheet (112) that has an upper surface that undergoes a silicon treatment (111) and a lower surface that has a smooth, shiny finish, wherein the lower surface of the second polypropylene sheet (109) with the security printing (110) is in contact with the upper surface of the third polypropylene sheet (112) of the base layer.

12. The method for manufacturing the self-adhesive sticker according to claim 11, characterized in that the method further comprises the steps of:
preparing a graphite plate (105) by adding silicone onto a sheet of mechanical paper to form a sheet of siliconed paper, rubbing a ground, dry, talcum-like graphite powder onto the siliconed paper to produce a static charge so that the graphite adheres to and forms the graphite plate (105) on the siliconed paper; and
laminating the graphite plate (105) between the protective layer and the central layer.

13. The method for manufacturing the self-adhesive sticker according to claim 11, characterized in that the method further comprises the steps of:
preparing a granulated plate by adding an adhesive layer to a sheet of uncoated paper and a finely ground, talcum-like pumice stone powder is sprayed onto the glued paper by means of a compressed air sprayer to produce a complete coating of granulated pumice stone powder sticking to the glued paper, and
laminating the granulated plate between the protective layer and the central layer.

14. A kit for preparing a self-adhesive sticker with superposed layers and security seal for fingerprinting and DNA collection according to claim 1, characterized in that it comprises the following layers: a paper layer (101) of preferably supercalendered, high-resistance kraft paper with a surface for printing text (100), a first polypropylene layer (102), a silicone lamination (103) having striping (104) on one of the lamination's (103) surface with no adhesive on one of the smaller lateral borders, a graphite or granulated plate layer (105), an acrylate adhesive lamination (106) having striping (107) with no adhesive on one of the smaller lateral borders applied to one surface of the lamination (106), a second polypropylene layer (108), an acrylate adhesive lamination (109) to be applied on one surface of the second polypropylene layer (108), an adhesive lamination (110) with security imprint, a silicone lamination (111), and a third polypropylene layer (112) with a smooth and shiny finish.
